(19) United States
(12) Patent Application Publication
(10) Pub. No.: US 2026/0048028 A1
(43) Pub. Date: Feb. 19, 2026

(54) Methods of Use for Disulfiram and Metabolites Thereof

(71) Applicant: PS Therapy, Inc., Westmont, IL (US)

(72) Inventor: SIDDHARTH NARENDRAN, COIMBATORE TAMIL NADU (IN)

(21) Appl. No.: 19/103,899

(22) PCT Filed: Aug. 15, 2023
(86) PCT No.: PCT/US23/30213

§ 371 Date: Feb. 14, 2025

Related U.S. Application Data
(60) Provisional application No. 63/398,518, filed on Aug. 16, 2022.
(60) Provisional application No. 63/399,321, filed on Aug. 19, 2022.

(51) Int. Cl. A61K 31/145; A61K 9/00; A61P 27/02; A61P 29/00

(52) U.S. Cl. CPC A61K 31/145; A61K 9/0048; A61P 27/02; A61P 29/00

(57) ABSTRACT

The present invention is directed to methods of treating ophthalmic conditions comprising administration of disulfiram or a metabolite of disulfiram selected from the group consisting of sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof.

### FIELD OF THE INVENTION

The present invention is directed to methods of treating ophthalmic conditions comprising administration of disulfiram or a metabolite of disulfiram selected from the group consisting of sodium diethyldithiocarbamate, copper diethyldithiocarbamate, zinc diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof.

### BACKGROUND OF THE INVENTION

Inflammation of the eye is a major cause of many ophthalmic conditions. Inflammation is the body's innate response to trauma and usually follows physical trauma or other pathologies that compromise cell integrity. Eye inflammation occurs in simple conditions such as dry eye and in more serious conditions such as macular degeneration and retinopathy. Inflammation is also associated with external aggravations such as surgery and infections.

Keratitis, or inflammation of the cornea, is a serious condition that may lead to permanent blindness. Keratitis may be caused by infections from several pathogens including amoebas, bacteria, viruses, and fungi. Fungal keratitis is a particularly invasive and serious form of keratitis and if not treated quickly often results in detrimental outcomes. In fact, from 26% to 63% of patients experience severe vision loss following a fungal infection of the cornea. Fungal keratitis occurs worldwide and is increasing in frequency, especially in developing countries where eye injuries are common, such as India.

Fungal keratitis normally occurs after a physical trauma to the eye or an ocular surface disease in which the epithelial layer is compromised. Fungi capable of infecting the cornea include fungi from genera including Fusarium, Aspergillus, Candida, Curvularia and Cladosporium. In warmer climates Aspergillus and Fusarium infections are more common. Current treatment for these fungal infections includes polyene anti-fungal agents such as amphotericin b and natamycin and less commonly triazole anti-fungal agents such as voriconazole and posaconazole and azoles such as miconazole, clotrimazole, ketoconazole and flucanzole.

While the anti-fungal treatments can reduce and eventually eliminate the fungal load in the eye, the treatments do not reduce the body's innate inflammation response used to combat the infection. The inflammation response itself causes serious damage to the corneal layer of the eye by destroying the cells of the cornea leading to scarring and eventual opacification. The inflammation response can also cause scarring of the conjunctival layer. However, reduction of inflammation is usually achieved using steroids, which themselves are a principal cause for developing fungal keratitis. The inability to effectively reduce inflammation compounded by the lack of early diagnosis is the likely cause of high incidence of severe vision loss associated with fungal keratitis.

Both physical trauma and infectious diseases of the eye such as fungal infections can lead to neovascularization. This neovascularization can be invasive and disturb the normal functioning of the retina including the macula leading to permanent blindness.

Diethyldithiocarbamate is the primary metabolite of disulfiram. Sodium diethyldithiocarbamate was studied extensively in the 1980s for treatment of human immunodeficiency virus. Hersh et al. Ditiocarb sodium (diethyldithiocarbamate) therapy in patients with symptomatic HIV infection and AIDS. A randomized, double-blind, placebo-controlled, multicenter study, JAMA, 1991 Mar. 27, 265(12):1538-44. Metal dithiocarbamate complexes such as zinc, copper, lead, nickel and cadmium-diethyldithiocarbamate complexes, which contains 2 or more diethyldithiocarbamates formed around a metal ion, have been studied as fungicides. Rathore et al., Synthesis, characterization and fungicidal activity of zinc diethyldithiocarbamate and phosphate, J Therm Ana Calor, 2007, 90:3, 681-686 and Rathore et al. Fungicidal and bactericidal activity of metal diethyldithiocarbamate fungicides: Synthesis and characterization, J Therm Ana Calor, 2008, 94:1, 75-81. However, there is no indication that simple diethyldithiocarbamate structures such as sodium diethyldithiocarbamate have any fungicidal effect.

Thus, there is a need in the art for an anti-inflammation treatment for the eye that does not lead to further pathophysiology. Further, there is need in the art for a fungal keratitis treatment that can not only reduce fungal load but also reduce inflammation of the cornea and surrounding tissues of the eye.

### SUMMARY OF THE INVENTION

The present invention is directed to methods of treating an ophthalmic condition comprising administering disulfiram or a metabolite of disulfiram selected from the group consisting of sodium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

The present invention is directed to methods of treating an ophthalmic condition comprising administering sodium diethyldithiocarbamate (“NaDDC”) or hydrates thereof to an eye of a subject in need thereof.

The present invention is further directed to reducing or ameliorating inflammation comprising administering disulfiram or a metabolite of disulfiram selected from the group consisting of sodium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

The present invention is further directed to reducing or ameliorating inflammation comprising administering sodium diethyldithiocarbamate or a hydrate thereof to an eye of a subject in need thereof.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A, 1B, 1C and 1D. Slit-lamp images of the cornea following alkali burn treatment for control group 1(FIG. 1A), prednisolone treated group 3 (FIG. 1B) and NaDDC treated group 2 (FIGS. 1C and 1D).

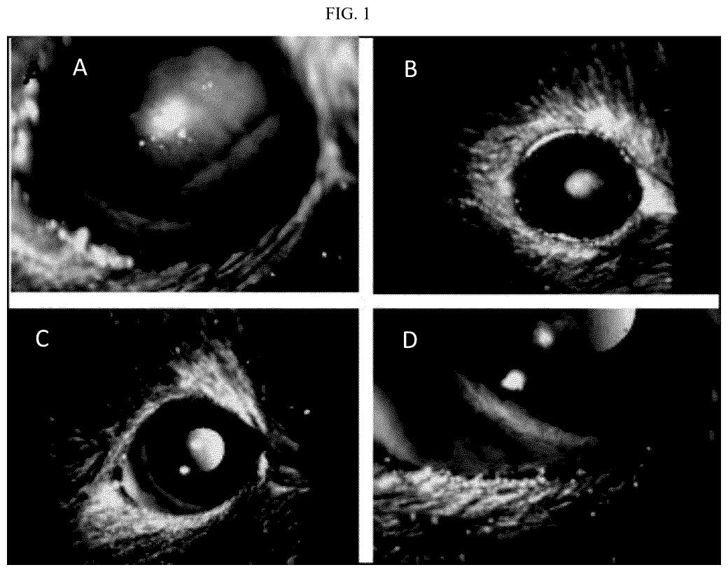

### DETAILED DESCRIPTION OF THE INVENTION

The Applicant has surprisingly discovered that sodium diethyldithiocarbamate (“NaDDC”) reduces inflammation of the eye. The Applicant has also surprisingly discovered that NaDDC treats fungal infections of the eye.

In one embodiment, the present invention is directed to methods of treating an ophthalmic condition comprising administering an effective amount of disulfiram or a metabolite of disulfiram selected from the group consisting of sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

In another embodiment, the present invention is directed to methods of treating an ophthalmic condition comprising administering an effective amount of NaDDC or a hydrate thereof to an eye of a subject in need thereof.

In a preferred embodiment, the ophthalmic condition is selected from the group consisting of infection, neovascularization, dry eye, fibrosis, keratitis, macular degeneration, diabetic retinopathy conjunctivitis, allergic eye disease, blepharitis, meibomian gland dysfunction, chemical and thermal burns, Sjogren syndrome, ocular cicatricial pemphigoid, Steven Johnson syndrome, corneal ulcers, uveitis, and thyroid eye disease and post-operative inflammation. Keratitis may be caused by fungal, viral or bacterial pathogens. In a more preferred embodiment, the ophthalmic condition is fungal keratitis. Treatment of fungal keratitis may comprise reduction of the fungal load.

Infections, fibrosis and neovascularization that may be treated by methods of the present invention include, but are not limited to, infections, fibrosis and/or neovascularization of the ocular surface, intraocular region and extra orbital region of the eye. Ocular surfaces include, but are not limited to, cornea, conjunctiva, sclera, episcleral and tenon capsule. Intraocular region includes, but is not limited to, uveal tissue, iris, choroid, retina, lens and optic nerve. Extra orbital regions include, but are not limited to, muscles, fat, skin and subcutaneous tissue of the extra orbital region and includes eyelids, lacrimal gland and tear drainage apparatus.

In another preferred embodiment, treatment of the ophthalmic condition comprises treatment of one or more symptoms of the ophthalmic condition. In a more preferred embodiment, the one or more symptoms comprise inflammation. In another more preferred embodiment, the one or more symptoms comprise corneal scarring and conjunctival scarring.

In another preferred embodiment, the present invention is directed to methods of reducing or ameliorating inflammation comprising administering an effective amount of disulfiram or a metabolite of disulfiram selected from the group consisting of sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

In another preferred embodiment, the present invention is directed to methods of reducing or ameliorating inflammation comprising administering an effective amount of NaDDC or a hydrate thereof to an eye of a subject in need thereof.

Inflammation that may be treated by methods of the present invention may occur in, but is not limited to, the ocular surface, intraocular region and extra orbital region of the eye. Ocular surfaces include, but are not limited to, cornea, conjunctiva, sclera, episcleral and tenon capsule. Intraocular region includes, but is not limited to, uveal tissue, iris, choroid, retina, lens and optic nerve. Extra orbital regions include, but are not limited to, muscles, fat, skin and subcutaneous tissue of the extra orbital region and includes eyelids, lacrimal gland and tear drainage apparatus.

In a more preferred embodiment, inflammation is caused by a fungal infection.

In an even more preferred embodiment, inflammation is caused by a fungal infection selected from the group consisting of a Fusarium spp., Aspergillus spp. and a Candida spp. infection. In an even more preferred embodiment inflammation is caused by an Aspergillus flavus infection.

In another preferred embodiment, the present invention is directed to treating or preventing scarring of the eye comprising administering an effective amount of disulfiram or a metabolite of disulfiram selected from the group consisting of sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof. In a more preferred embodiment, the scarring of the eye is selected from the group consisting of corneal scarring, conjunctival scarring and a combination thereof.

Administration of disulfiram or a metabolite of disulfiram selected from the group consisting of sodium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof may occur topically, orally, intravitreously as an injection or via an injectable implant, intracamerally, to the subtenon and other intraocular routes known in the art. Topical administration may occur via a spray or an ocular drop.

Disulfiram (CAS #97-77-8), also known as 1,1′-disulfanediylbis(N,N-diethylmethanethioamide), has the following chemical structure:

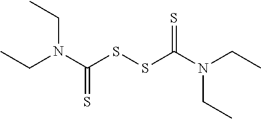 [SMILES: CCN(CC)C(=S)SSC(=S)N(CC)CC]

Sodium diethyldithiocarbamate (CAS #148-18-5), also known as sodium;N,N-diethylcarbamodithioate, has the following chemical structure:

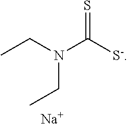

S-methyl N,N-diethylthiocarbamate (CAS #37174-63-3) also known as S-methyl N,N-diethylcarbamothioate has the following chemical structure

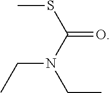

S-methyl N,N-diethylthiocarbamate sulfoxide (CAS #140703-15-7) has the following chemical structure

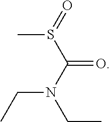

S-methyl-N,N-diethylthiocarbamate sulfone (CAS #155514-79-7) also known as N,N-diethyl-1-methylsulfonylformamide has the following chemical structure

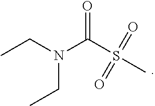

Diethyldithiocarbamate (CAS #392-74-5) also known as N,N-diethylcarbamodithioate has the following chemical structure

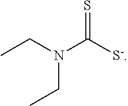

Methyl diethyldithiocarbamate (CAS #686-07-7) also known as methyl N,N-diethylcarbamodithioate has the following chemical structure

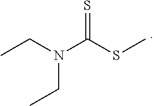

As used herein the term “treatment” or “treating” refers to reducing, alleviating or ameliorating the ophthalmic condition or one or more symptoms of the ophthalmic condition.

As used herein the term “effective amount” refers to the amount necessary to treat a patient in need thereof.

Throughout the application, the singular forms “a,” “an,” and “the” include plural reference unless the context clearly dictates otherwise.

Throughout the application, all disclosed ranges include all possible values within those ranges. All possible values within the ranges disclosed in the application can also be used as endpoints for additional ranges between these values.

The disclosed embodiments are simply exemplary embodiments of the inventive concepts disclosed herein and should not be considered as limiting unless the claims expressly state otherwise.

The following example is intended to illustrate the present invention and to teach one of ordinary skill in the art how to use the methods of the invention. The example is not intended to be limiting in any way.

### Examples

### Example 1—Inhibition of Inflammation Marker IL-8

Interleukin-8 (“IL-8”) is a known marker for ocular inflammation. Ghasemi et al. Roles of IL-8 in ocular inflammations: a review, Ocul Immunol Inflamm 2011 Dec;19(6):401-12.

### Method

3 sets of human corneal epithelial (“HCE”) cells were plated and assayed for IL-8 mRNA levels as a control. 9 sets of HC cells were then infected with 5×10⁷ milliliters of Aspergillus flavus spores. 16 hours post infection the cells were assayed for IL-8 mRNA levels. 3 sets of HCE cells were then administered DSF in DMSO. Several hours following administration of each, IL-8 mRNA levels were assayed. Results of this study can be found in Table 1, below.

TABLE 1

| | IL-8 mRNA levels | |
|---|---|---|
| | (% change from | IL-8 mRNA levels |
| | uninfected) | (% reduction from water) |
| Water/Carrier | 183.9 | 0 |
| DSF | 21.2 | 88.5 |

### Results

As demonstrated in Table 1, disulfiram (“DSF”) reduced the percent of IL-8 mRNA levels induced by A. flavus infection in human corneal epithelial cells by 88.5% to an absolute level of 21.2% over uninfected cells.

### Example 2—Inhibition of Inflammation Marker IL-8

### Method

3 sets of HCE cells were plated and assayed for IL-8 mRNA levels as a control. 9 sets of HC cells were then infected with 5×10⁷ milliliters of Aspergillus flavus spores. 16 hours post infection the cells were assayed for IL-mRNA levels. 3 sets of HCE cells were then each administered NaDDC in water. Several hours following administration of each, IL-8 mRNA levels were assayed. Results of this study can be found in Table 2, below.

TABLE 2

| | IL-8 mRNA levels | IL-8 mRNA levels |
|---|---|---|
| | (% increase from uninfected) | (% reduction from water) |
| Water | 70.9 | 0 |
| NaDDC | 10.5 | 85.19 |

### Results

As demonstrated in Table 2, NaDDC reduced the percent of IL-8 mRNA levels induced by A. flavus infection in human corneal epithelial cells by 85.19% to an absolute level of only 10.5% over uninfected cells. Thus, NaDDC is capable of significantly reducing inflammation caused by fungal infections of human corneal epithelial cells.

### Example 3—Inhibition of Inflammation Markers IL-8 and IL-6

### Method

Sets of HCE cells were plated in iso-osmotic solutions (290 milliosmoles) having an and assayed for IL-8 and IL-6 mRNA levels as a control. Sets of HC cells were subjected to hyperosmolarity conditions (i.e. placed in a solution having an osmolarity of 400 milliosmoles) either with or without NaDDC. 6 hours post hyperosmolarity induction the cells were assayed for IL-mRNA levels. Results of this study can be found in Table 3, below.

TABLE 3

| | IL-8 mRNA | IL-6 mRNA | IL-8 mRNA | IL-6 mRNA |
|---|---|---|---|---|
| | levels | levels | levels | levels |
| | (% increase | (% increase | (% decrease | (% decrease |
| | from −control) | from −control) | from +control) | from +control) |
| 290 milliosmoles | — | — | — | — |
| (Negative Control) | | | | |
| 400 milliosmoles | 1500 | 900 | — | — |
| (Positive Control) | | | | |
| 400 milliosmoles + | 0 | 100 | 1500 | 450 |
| NaDDC | | | | |

### Results

As demonstrated in Table 3, NaDDC reduced the percent of IL-8 mRNA levels of cells induced by hyperosmolarity by 1500% to an absolute level of equal to that of iso-osmotic cells. Further, NaDDC reduced the percent of IL-6 mRNA levels of cells induced by hyperosmolarity by 450% to an absolute level 100% more than that of iso-osmotic cells. Thus, NaDDC is capable of significantly reducing inflammation caused by hyperosmolarity in human corneal epithelial cells.

### Example 4—Therapeutic Evaluation of NaDDC in Alkali-Burn Injury Treatment in Mice

### Methods

### Experimental Design

C57BL/6 mice were divided into three groups, control group 1 (n=6), group 2 (n=6) and group 3 (n=3). Each group consisted of mice with their left eye (“LE”) left undisturbed as a control, while the right eye (“RE”) of all mice in each group was subjected to alkali burn. Prior to inducing alkali burn, mice were anesthetized using an injection of an anesthetic cocktail containing ketamine (20 mg/ml) and xylazine (4 mg/ml) at a dose of approximately 100 l per 25 g mouse. Topical analgesia was provided using proparacaine hydrochloride (0.5%) on the corneal surface. A round filter paper piece (˜2 mm in diameter) was soaked in a 0.5 M NaOH solution. The NaOH-soaked filter paper was gently placed on the central cornea of the RE for precisely 30 seconds, generating an acute alkali burn. Following the removal of the filter paper, the RE was flushed gently with 10 ml of 1×PBS using a 10 ml syringe to ensure thorough removal of residual NaOH. Immediately after the eye flush, the treatment application was initiated for Group 2 and Group 3 mice. Group 1 was left untreated. For Group 2, a drop of 300 μM NaDDC was topically applied to the cornea of the RE. For Group 3, the RE was topically treated with commercially available prednisolone. The treatment was repeated three times daily for a duration of 7 days.

### Clinical Evaluation

On the 7^{th} day following the alkali burn, corneal images were captured using slit microscopy to monitor changes in corneal opacity and neovascularization. The degree of corneal injury was evaluated and scored in a blinded manner by trained observers. Corneal opacity was evaluated using a grading scale ranging from 0 to 4, where 0 represented a completely clear cornea, 1 indicated slight haziness with the iris and pupil easily visible, 2 denoted slight opacity with detectable iris and pupil, 3 indicated significant opacity with difficulty in detecting pupils, and 4 represented complete opacity with no view of the pupil. The degree of neovascularization was scored on a scale of 0 to 3, with 0 indicating no new vessels, 1 representing new vessels present at the corneal limbus, 2 denoting new vessels extending from the corneal limbus toward the corneal centre, and 3 indicating new vessels spanning the entire corneal centre. At the end of the treatment period, mice from control group 1 and NaDDC treated group 2 were sacrificed and the eyes were enucleated for further analysis, including RT-PCR studies, to investigate the expression levels of tumor necrosis factor alpha (“TNFα”), gasdermin D (“GSDMD”), intercellular adhesion molecule 1 (“ICAM1”) and transforming growth factor beta 1 (“TGFβ1”).

### Results

Corneal opacity and neovascularization were evaluated by scoring slit lamp images of the mice in the different experimental groups. The results show that in the control group 1, corneal opacity scores ranged from 1 to 4, with a mean score of 2.33. However, the treatment with NaDDC (group 2) significantly reduced corneal opacity scores compared to the control group 1, with a mean score of 0.83. The treatment with prednisolone (group 3) showed a corneal opacity mean score of 2.67. Regarding neovascularization, the control group showed a mean score of 0.8. Both the NaDDC and prednisolone treatments resulted in a complete absence of neovascularization with all mice scoring 0.

Expression levels for each of TNFα, GSDMD, ICAM1, and TGFβ1 in NaDDC treated mice were reduced compared to the untreated control group. Specifically, the expression level of TNFα in mice in the NaDDC treated group was only 8% that of mice in the untreated control group; GSDMD expression was only 42% that of the control group, ICAM1 was only 5% that of the control group and TGFβ1 was only 28% that of the control group.

This reduction in each of TNFα, GSDMD, ICAM1, and TGFβ1 expression levels in the NaDDC treatment group indicates that NaDDC reduced inflammation caused by the alkali burn treatment.

What is claimed is:

1. A method of treating an ophthalmic condition comprising administering an effective amount of disulfiram or a metabolite of disulfiram sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

2. The method of claim 1, wherein a metabolite of disulfiram is administered.

3. The method of claim 1, wherein sodium diethyldithiocarbamate or a hydrate thereof is administered.

4. The method of claim 1, wherein administration occurs topically.

5. The method of claim 1, wherein treatment comprises treating one or more symptoms of the ophthalmic condition.

6. The method of claim 5, wherein the one or more symptoms comprise inflammation.

7. The method of claim 1, wherein the ophthalmic condition is selected from the group consisting of infection, neovascularization, dry eye, fibrosis, fungal keratitis, bacterial keratitis, macular degeneration, diabetic retinopathy and post-operative inflammation.

8. The method of claim 7, wherein the ophthalmic condition is fungal keratitis.

9. The method of claim 8, wherein fungal load is reduced or ameliorated.

10. The method of claim 9, wherein inflammation is reduced or ameliorated.

11. A method of reducing or ameliorating inflammation comprising administering an effective amount of disulfiram or a metabolite of disulfiram sodium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

12. The method of claim 11, wherein a metabolite of disulfiram is administered.

13. The method of claim 11, wherein sodium diethyldithiocarbamate or a hydrate thereof is administered.

14. The method of claim 11, wherein administration occurs topically.

15. The method of claim 11, wherein the inflammation is caused by a fungal infection.

16. The method of claim 15, wherein the fungal infection is Aspergillus flavus.

17. A method of treating or preventing scarring of the eye comprising administering an effective amount of disulfiram or a metabolite of disulfiram selected from the group consisting of sodium diethyldithiocarbamate, copper diethyldithiocarbamate, zinc diethyldithiocarbamate, lead diethyldithiocarbamate, nickel diethyldithiocarbamate, cadmium diethyldithiocarbamate, S-methyl-N,N-diethylthiocarbamate, S-methyl N,N-diethylthiocarbamate sulfoxide, S-methyl-N,N-diethylthiocarbamate sulfone, diethyldithiocarbamate and methyl diethyldithiocarbamate and hydrates thereof to an eye of a subject in need thereof.

18. The method of claim 17, wherein a metabolite of disulfiram is administered.

19. The method of claim 17, wherein sodium diethyldithiocarbamate or a hydrate thereof is administered.

20. The method of claim 17, wherein the scarring of the eye is selected from the group consisting of corneal scarring, conjunctival scarring and a combination thereof.
